(19) United States
(12) Patent Application Publication
(10) Pub. No.: US 2014/0018717 A1
(43) Pub. Date: Jan. 16, 2014

(54) NOVEL MEDICAL COUNTERMEASURE FOR FIRST RESPONDER USE IN MASS CASUALTY THERMAL AND/OR RADIOLOGICAL BURN INJURY EVENT

(71) Applicant: Bio Med Sciences, Inc., Allentown, PA (US)

(72) Inventor: Mark E. Dillon, Center Valley, PA (US)

(21) Appl. No.: 13/937,784

(22) Filed: Jul. 9, 2013

Related U.S. Application Data
(60) Provisional application No. 61/669,961, filed on Jul. 10, 2012.

(51) Int. Cl. A61F 13/00

(52) U.S. Cl. CPC A61F 13/00063
USPC 602/48; 427/2.31

(57) ABSTRACT

A wound dressing for mass casualty burn injury events is provided that is capable of being stockpiled for long periods of time without special storage conditions, may be applied by persons with little or no training, immediately restores skin barrier function and reduces pain, manages wound exudate, accommodates edema, and is transparent. The dressing does not integrate into the wound while simultaneously providing antimicrobial activity, thereby avoiding complication of the wound condition when proper medical attention is delayed for significant periods of time.

### BACKGROUND OF THE INVENTION

1. Field of the Invention

This invention relates to a novel wound dressing design. Particularly, this invention relates to a wound dressing which incorporates a unique set of features ideally suited for use by first responders in a mass casualty thermal and/or radiological burn injury event. A particular embodiment of this invention may be economically mass-produced, has an long or indefinite shelf life, requires no special storage conditions, is not temperature sensitive, can be supplied in rolls, can be easily applied by persons with little or no training, immediately restores skin barrier function, provides an antimicrobial effect, reduces pain, manages wound exudate, accommodates edema, is transparent so that wounds may be visualized (e.g., seen) without dressing removal, and will not complicate the wound condition when proper medical attention is delayed for significant periods of time.

2. Description of the Prior Art

In the field of woundcare there exist several general categories of commonly used dressings. Each type of dressing has its advantages and disadvantages, and is indicated for certain wound conditions and user preferences.

Some dressings aggressively adhere to the wound surface. For example, conventional gauze integrates into the wound as healing occurs and eschar forms on the wound surface. Other types of dressings are designed to adhere to the surrounding intact tissue around the wound site, but not directly to the wound. Examples of this type of dressing include polyurethane films coated with acrylic pressure sensitive adhesive. Other types of dressings are designed to be substantially nonadherent. Examples of this type include polyethylene oxide hydrogels, but also non-hydrogel materials such as that described in my U.S. Pat. No. 4,832,009, which is incorporated herein by reference. The latter example is a dressing made from an interpenetrating polymer network (“IPN”) of polytetrafluoroethylene (“PTFE”) and silicone, and is presently marketed by Bio Med Sciences, Inc. of Allentown, Pa. as Silon®.

Many commercially available dressings incorporate antimicrobial substances to reduce or prevent infections. Typical examples of such antimicrobials include various ionic forms of silver, drugs such as Polymyxin B Sulfate, Bacitracin Zinc, Neomycin, or combinations thereof. In each case the active ingredient is delivered to the wound and is depleted from the dressing over time.

There are a wide variety of wound types. The terms “first,” “second” and “third” degree are often used to describe the extent of the injury, particularly for burn injuries. First degree wounds involve only the epidermis or outermost layer of skin. A mild to moderate sunburn is a good example; the surface of the skin is not breached, there is no bleeding and no chance of infection. A second degree, or partial thickness injury, extends through the epidermis and into the dermis. As long as part of the dermis remains, the epidermis will regenerate and the wound will spontaneously heal if proper conditions are maintained. Failure to maintain proper conditions results in delayed healing and may even cause a partial thickness wound to convert to full thickness via infection and/or integration of the dressing into the wound.

A full thickness, or third degree injury, extends entirely through the dermis to the subdermal tissue. These wounds will not spontaneously heal because dermal tissue is missing and cannot generate and support epidermal tissue. In such cases a tissue transplant is required by harvesting intact skin from a donor site or the application of a biosynthetic skin substitute. In the former case a partial thickness autograft is taken so that a portion of the dermal layer is transplanted but a portion remains behind, thereby allowing both sites to heal. In the later case, modern technology has provided several alternatives to reduce the need for donor tissue. Such products may provide a manufactured dermal base or cultured epithelial surface, but each is biologically derived.

Such products, however, are not without their own drawbacks. Commercial products such as Integra® (Integra LifeSciences, Inc., Plainsboro, N.J.) and TransCyte® (Advanced Biohealing, Westport, Ct.) require a high degree of expertise to apply and manage during the healing process. Biobrane® (Smith & Nephew Company, London, UK) contains a nylon fabric that is woven from tri-filament threads and covalently bonded with collagen peptides from a porcine dermal collagen source. The multiple filaments provide a high exposure to the wound surface resulting in an increased adherence to the wound. Because of its adhesive nature, Biobrane in particular requires a high degree of skill and continuing care to avoid wound complications. Additionally, these products, like all materials containing biological components, are readily degradable and usually require special storage conditions such as refrigeration. This leads to inherently short shelf lives.

In a mass casualty thermal and/or radiologic burn injury event, such as those modeled by Bell & Dallas in their paper titled “Vulnerability of populations and the urban health care systems to nuclear weapon attack—examples from four American cities,” many challenges would immediately arise in managing large numbers of patients, particularly in view of the complications of ionizing radiation occurring concurrently.

The first challenge is that burn treatment is a highly specialized form of medical care, which is the reason why designated “burn centers” exist all around the world. In the United States there are approximately 1,500 “burn beds,” in about 100 specialized facilities to treat burn patients. Of those, approximately 1,000 are occupied at any particular time. Consequently the resources needed to treat a large number of burn patients, hundreds or perhaps thousands, would immediately and completely overwhelm existing capacity. Logistically, the only course of action would be to transport and admit patients to conventional, non-specialized facilities for interim care until proper burn treatment can be provided. Furthermore, local resources and infrastructure could be significantly compromised thereby delaying the ability to mobilize and transport patients. Victims could be waiting hours or even days in a crisis zone before trained medical personal are available. For burn patients this is a life threatening and critical issue. Skin barrier function must be immediately restored and infection must be prevented if there is any hope of stabilization and eventual survival.

According to Mosteller R D. Simplified calculation of body-surface area. N Engl J Med 1987;317:1098, the average adult male has a total body surface area of approximately 1.9 square meters. So ideally, a mass casualty burn dressing would be delivered in conveniently transported sterilized units of enough material to cover that amount of surface area.

The countermeasure required to stabilize patients in such a scenario must be a wound dressing that is easily stockpiled, transported, and applied by people with little or no training. Furthermore, the dressing must not complicate the wound by allowing infection or wound adherence/integration to occur. The dressing must also accommodate often copious amounts of fluid or exudate produced by such wounds. Importantly, the dressing must also be elastic and flexible to accommodate the dramatic edema or swelling that occurs after thermal burn injury; else the patient may suffer compartmental syndrome when the compression of nerves, blood vessels, and muscle leads to tissue death from lack of oxygenation. Ideally the dressing would also be transparent and not require changes, i.e. a single application would suffice.

### SUMMARY OF THE INVENTION

I have unexpectedly discovered that a carefully designed silicone-PTFE IPN in conjunction with a silane-based antimicrobial surface treatment provides a wound dressing ideally suited for use in a mass casualty thermal and/or radiological burn injury scenario.

A thin elastic film approximately 50 microns thick of silicone-PTFE IPN material supported by a paper carrier substrate was coated on one side with a tacky silicone formulation containing 5 percent by weight 3-methoxysilylpropydimethyloctadecyl ammonium chloride. The material was then passed through a tunnel style oven and cured. The roll of coated IPN on substrate was then passed through a die cutting apparatus to create small slits, or fenestrations, in the film approximately 2.5 mm long and spaced approximately 1.5 cm apart. The film was then removed from the carrier substrate and cut to approximately 20 cm widths and rewound onto itself into rolls approximately 10 meters long.

### BRIEF DESCRIPTION OF THE DRAWINGS

FIG. 1 shows a cross-sectional view of a preferred embodiment of the inventive dressing (40), constructed in accordance with the invention, having a layer (10) of an IPN material that is coated with a layer (20) of tacky silicone containing 3-methoxysilylpropydimethyloctadecyl ammonium chloride with fenestrations (30) cut in the dressing (40) at regular intervals.

FIG. 2 is a top plan view of the silicone-PTFE IPN dressing (40) shown in FIG. 1, showing the fenestrations (30).

FIG. 3 is a perspective view of the inventive dressing (40), constructed in accordance with the invention, wrapped onto a core (50) to form a roll (60) of the dressing (40).

FIG. 4 is a perspective view of the dressing (40) being applied to a thermal and/or radiological burn injury patient (70).

FIG. 5 is Chart 1 which is a Stress-Strain Plot for Standard Silon-TSR.

FIG. 6 is Chart 2 which is a 3-Log Reduction of E. coli in 2 Hours.

FIG. 7 is Chart 3 which is a 4-Log Reduction of E. coli in 2 Hours.

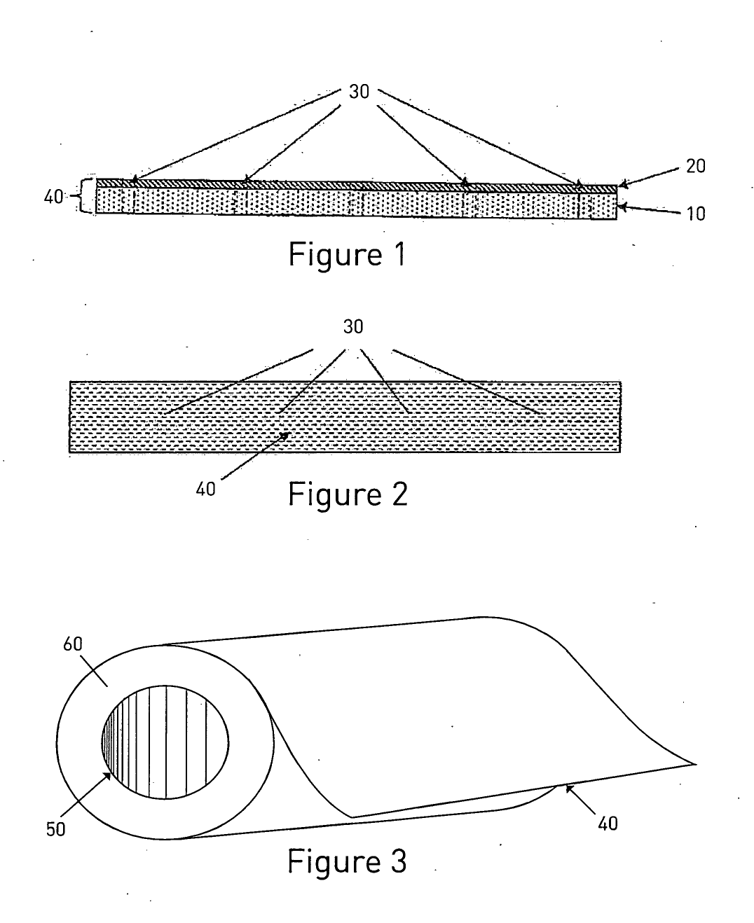

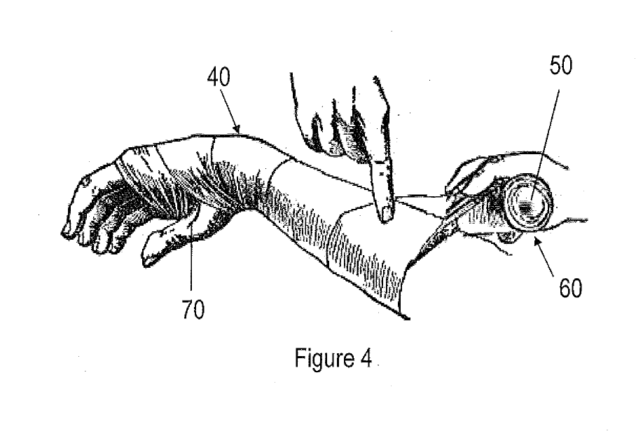

### DETAILED DESCRIPTION OF PREFERRED EMBODIMENTS

In a preferred embodiment of this invention, a wound dressing (40) comprises a 10 meter long sheet or layer (10) of a thin film of silicone-PTFE IPN material coated on one side with a coating (20) of a tacky silicone formulation containing 5 percent by weight 3-methoxysilylpropydimethyloctadecyl ammonium chloride. Preferably, the wound dressing (40) is fenestrated with 2.5 mm slits (30) preferably separated by approximately 1.5 cm from each other, and the wound dressing (40) preferably is cut to be 20 cm wide, and preferably the wound dressing (40) is self-wound onto a plastic core (50) with the tacky coating (20) wrapped “in” against the core (50).

3-methoxysilylpropydimethyloctadecyl ammonium chloride is particularly preferred because it readily bonds to a silicone-based substrate and it physically disrupts the cell membrane of the target organism (e.g., a germ) on contact. This means organisms do not metabolize the active agent and become resistant. Through extensive studies (including ISO 10993 standards), this colorless, non leaching material was found to be safe and effective against a broad spectrum of fungi, bacteria, algae and yeast. Because 3-methoxysilylpropydimethyloctadecyl ammonium chloride chemically bonds to a treated substrate of the invention, the dressing (40) itself becomes antimicrobial. This is an important difference from other commercially available antimicrobial dressings based on silver or other compounds which are delivered to the wound and are therefore depleted and lose effectiveness over time.

Both the Silon® dressing (that is, a Bio Med Sciences, Inc. dressing made from an interpenetrating polymer network (“IPN”) of polytetrafluoroethylene (“PTFE”) and silicone) and 3-methoxysilylpropydimethyloctadecyl ammonium chloride materials are chemically stable, showing shelf life in the range of 5 years or more. Data to date actually suggests an indefinite shelf life.

The above preferred embodiment is not intended to be limiting, as variations on the illustrated design would be obvious to those skilled in the art. For example, precut sheets on a release liner may be used instead of rolls. Furthermore, it may be advantageous to use silver or other antimicrobial compounds, or combinations of such with 3-methoxysilylpropydimethyloctadecyl ammonium chloride for clinically therapeutic reasons. Additionally, an antimicrobial agent may be incorporated into the non-wound contacting side of the dressing to reduce the overall bioburden of the site. By incorporating the antimicrobial agent into silicone-PTFE IPN material layer (10) microbes on the exterior surface of the wound site would be inhibited, thus improving the overall hygiene of the entire wound environment.

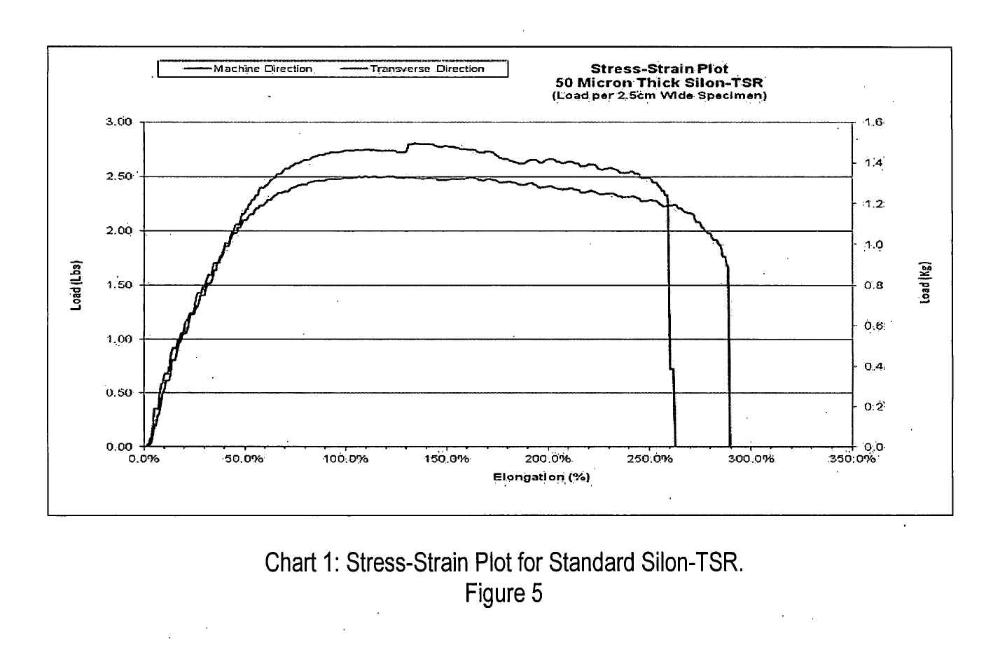

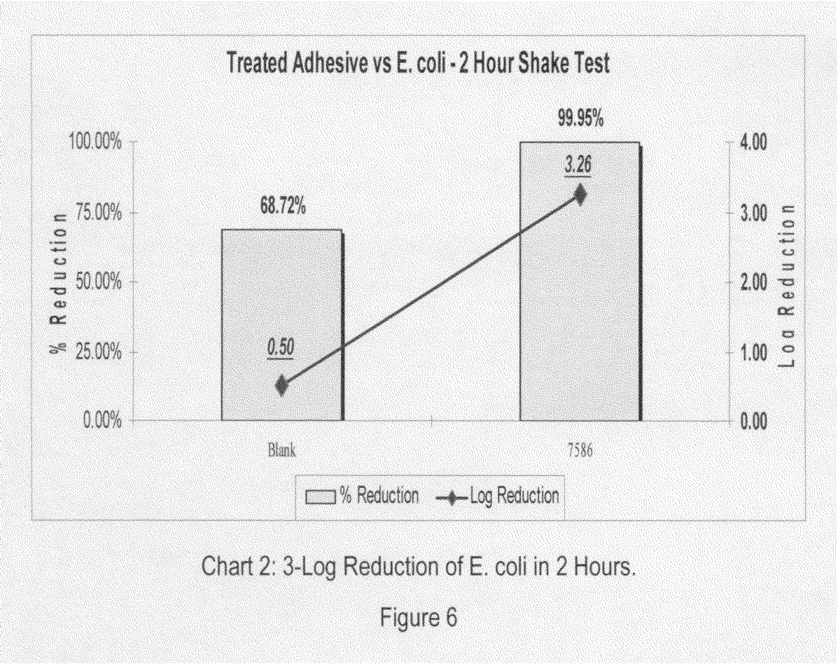

Lastly, the fenestration pattern itself may engineered to optimally accommodate edema by designing various cut-patterns to effect expansion characteristics or even provide built-in “break points” so that compartmental syndrome is avoided, although the elasticity of the IPN material of the inventive dressing (40) is expected to provide for sufficient expansion of the inventive dressing (40) to avoid compartmental syndrome. Chart 1 of FIG. 5 shows the remarkable elasticity of the basic silicone-PTFE IPN material without fenestrations.

The following example is not intended to be limiting, as minor variations on the described processes would be obvious to those skilled in the art. Likewise, it is believed that other materials could be used to achieve the same dressing design.

### EXAMPLE 1

A continuous sheet or layer (10), approximately 20 meters long and 40 cm wide, of silicone-PTFE IPN was manufactured according to established methods using a paper carrier substrate. The sheet or layer (10) of silicone-PTFE IPN film measured approximately 50 microns in thickness. The sheet or layer (10) of silicone-PTFE IPN film was then passed through a knife-over-roll assembly and coated with approximately 30 grams per square meter (gsm) of a silicone elastomer (product code 7-9600 from Dow Corning Corporation of Midland, Mich.), mixed with 5 percent by weight of 3-methoxysilylpropydimethyloctadecyl ammonium chloride (product code HM4100 from BIOSAFE, Inc. of. Pittsburgh, Pa.) to form a coating (20) of tacky silicone containing 3-methoxysilylpropydimethyloctadecyl ammonium chloride on the sheet or layer (10) of the silicone-PTFE IPN film.

Using a rotary die cutting apparatus, fenestrations (30), preferably approximately 2.5 mm long, were preferably cut into the dressing (40). Reconfiguring the rotary die cutting apparatus for slitting and rewinding, the fenestrated dressing (40) was slit to 20 cm wide and rewound onto 2.5 cm diameter cores (50) in lengths of 10 meters with the coated side of the dressing (40)(that is, the side of the dressing (40) having the coating (20)) in contact with the plastic core (50).

When fenestrations (30) are not provided to the dressing (40), the same manufacturing process set out above for manufacturing fenestrated wound dressing (40) may be used to manufacture non-fenestrated wound dressing (40), except that the step of using the rotary die cutting apparatus to cut fenestrations into the wound dressing (40) may be skipped.

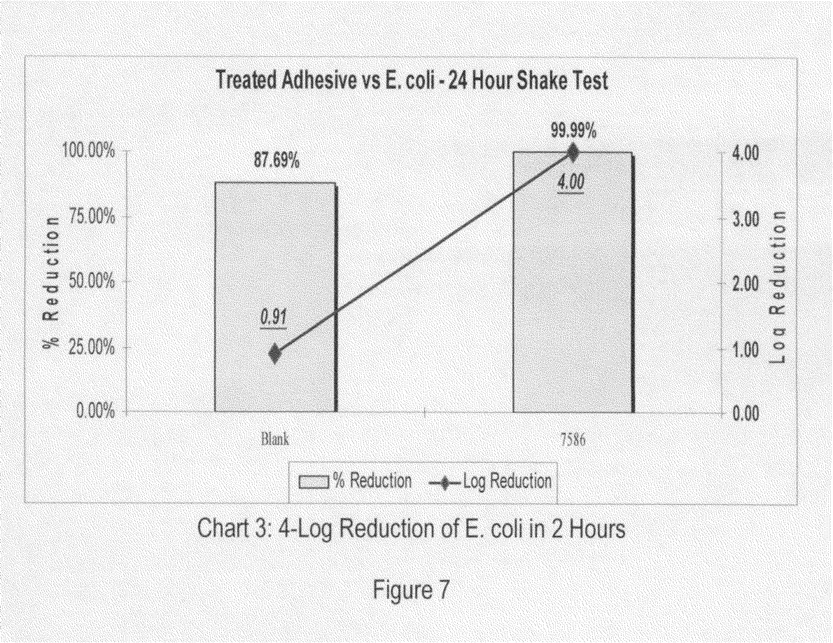

Samples of the product dressing (40) were tested using ASTM method E2149-01-Shake for E. coli, with results showing a 3-log reduction in 2 hours, and a 4-log reduction in 24 hours. See Charts 2 and 3 shown in FIGS. 6 and 7.

1) A wound dressing capable of being mass-produced and stockpiled comprising the following characteristics:
a) thin, and
b) conforming, and
c) elastic, and
d) antimicrobial, and
e) non-temperature sensitive, and
f) possessing a long shelf life, and
g) does not allow wound integration,
h) can be easily applied by persons with little or no training.

2) A method of manufacturing the wound dressing of claim 1 consisting of (1) producing a thin film of a polymer compound, (2) passing said thin film through a coating assembly and depositing a layer of a tacky antimicrobial substance, (3) converting said coated polymer film into usable shapes such as rolls or sheets.

3) A method of treating a cutaneous injury by applying the dressing of claim 1 to the wound surface.

4) A wound dressing for treating a cutaneous injury, comprising
a layer of thin film of a polymer compound, and
a layer of a tacky antimicrobial substance adhered to the layer of thin film of a polymer compound.

5) The wound dressing of claim 4, the layer of film of a polymer compound being a silicone-PTFE IPN film.

6) The wound dressing of claim 4, the layer of tacky antimicrobial substance being a silicone elastomer mixed with an antimicrobial substance.

7) The wound dressing of claim 6, the antimicrobial substance being 3-methoxysilylpropydimethyloctadecyl ammonium chloride.
